(19) United States
(12) Patent Application Publication
(10) Pub. No.: US 2026/0070890 A1
(43) Pub. Date: Mar. 12, 2026

(54) MODULATORS OF TAU LIQUID-LIQUID PHASE SEPARATION AND METHODS THEREOF

(71) Applicant: JAWAHARLAL NEHRU CENTRE FOR ADVANCED SCIENTIFIC RESEARCH, BANGALORE (IN)

(72) Inventors: Govindaraju THIMMAIAH, BANGALORE (IN); Madhu RAMESH, BANGALORE (IN); Chenikkayala BALACHANDRA,, BANGALORE (IN)

(21) Appl. No.: 19/105,740

(22) PCT Filed: Aug. 22, 2023
(86) PCT No.: PCT/IN2023/050789

§ 371 Date: Feb. 21, 2025

(51) Int. Cl. C07D 401/14; A61K 31/497; A61P 25/28

(52) U.S. Cl. CPC C07D 401/14; A61K 31/497; A61P 25/28

(57) ABSTRACT

The present disclosure provides a compound of Formula (I), its stereoisomers, intermediates, or pharmaceutically acceptable salt thereof and a method of preparation of the same. In addition, the present disclosure provides a pharmaceutical composition comprising the compound of Formula (I) and its stereoisomers, intermediates, or pharmaceutically acceptable salt thereof; a pharmaceutically acceptable carrier; and optionally, in combination with one or more other pharmaceutical compositions. Further, the present disclosure provides a method for prevention or treatment of neurodegenerative disease or for treatment of a condition mediated by aggregation of tau, by administering an effective amount of the compound of Formula (I), its stereoisomers, intermediates, pharmaceutically acceptable salt thereof, or the pharmaceutical composition; a pharmaceutically acceptable carrier; and optionally, in combination with one or more other pharmaceutical compositions or with a clinically relevant immune modulator agent.

### FIELD OF INVENTION

The present disclosure broadly relates to the field of medicinal chemistry. In particular, the present disclosure relates to the development of protein LLPS (liquid-liquid phase separation) modulating compounds of Formula I, its stereoisomers, intermediates, or pharmaceutically acceptable salts thereof. The subject matter further relates to a field of drugs for treating the disorders related to the nervous system and in particular relates to a pharmaceutically active compound for use in the treatment of neurodegenerative diseases and neuroinflammatory disorders.

### BACKGROUND OF THE INVENTION

Dementia and cancer are the two major health and socioeconomic burdens worldwide that need immediate attention. Dementia is a spectrum of disorders, most of them characterized by the accumulation of protein aggregates. Liquid-liquid phase separation is a complex phenomenon that plays a significant role in cellular physiology and disease conditions like cancer and neurodegenerative disorders.

Liquid-liquid phase separation (LLPS) of proteins and other biomolecules in cells results in the formation of membraneless compartmentalisation that is in dynamic exchange with the surroundings (Hyman A A et al., Liquid-Liquid Phase Separation in Biology. Annu. Rev. Cell Dev. Biol. 2014; 30:39-58). The LLPS is mediated by multivalent, weak, dynamic, and promiscuous interactions among biomolecules, majorly proteins and nucleic acids (Bentley E P et al., Physical Chemistry of Cellular Liquid-Phase Separation. Chemistry (Weinheim an der Bergstrasse, Germany). 2019; 25 (22): 5600-5610.). LLPS results in the formation of processing-bodies (P-bodies), Cajal bodies, nucleolus, super enhancers, chromatin organisation, and stress granules to regulate different physiological functions like gene regulation, stress response, synaptic signalling, and immune responses. Further, LLPS play a detrimental role in inducing disease conditions like cancer, inflammatory diseases, viral infections, and neurodegenerative disorders. Many proteins with structural flexibility and multivalent interactions undergo LLPS in healthy and disease conditions. FUS, α-Syn, TDP-43 and tau associated with various neurodegenerative disorders have been shown to undergo LLPS from solution to liquid droplet condensates and insoluble aggregates that are toxic to cells. The protein concentration in the phase separated droplet is higher with low molecular motion that promotes aggregation to form toxic species responsible for neurodegenerative disorders.

The protein expression associated with cancer and the aggregation of proteins implicated in neurodegenerative disorders was thus regulated through LLPS. The critical role of LLPS in disease conditions underscores the protein LLPS as a potential therapeutic target. The failure in the drug discovery for AD (Alzheimer's disease) and other dementia conditions, the development of resistance to drugs available for cancer treatment emphasize looking for tangible alternatives. The chemical tools that modulate LLPS are limited and drug like small molecules are scarce. Therefore, there is an unmet need to establish an approach to develop small molecules that effectively modulate protein LLPS.

### SUMMARY OF THE INVENTION

In a first aspect of the present disclosure, there is provided a compound of Formula (I),

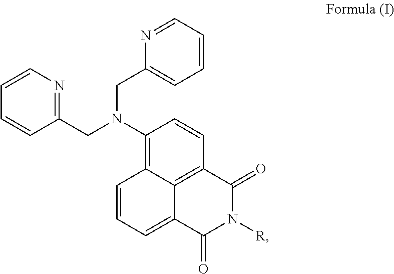

stereoisomers, intermediates, or pharmaceutically acceptable salts thereof,
wherein
R is C₁₋₁₀ alkyl substituted with a group selected from C₂₋₁₀ heterocyclyl, C₆₋₁₀ aryl, or C₂₋₁₀ heteroaryl, wherein C₂₋₁₀ heterocyclyl, C₆₋₁₀ aryl, or C₂₋₁₀ heteroaryl is optionally substituted with one or more substituents selected from hydrogen, hydroxyl, carboxyl, oxo, CONH₂, NH₂, C₁₋₁₀ alkyl, C₁₋₆ alkylamine, halogen, C₁₋₆ haloalkyl, cyano, or R¹,
R¹ is selected from C₁₋₁₀ alkyl, C₁₋₆ alkylamine, or C₁₋₆ haloalkyl, wherein C₁₋₁₀ alkyl, C₁₋₆ alkylamine, or C₁₋₆ haloalkyl is optionally substituted with C₆₋₁₀ aryl or C₂₋₁₀ heteroaryl, and wherein C₆₋₁₀ aryl or C₂₋₁₀ heteroaryl is optionally substituted with one or more substituents selected from hydroxyl, carboxyl, oxo, CONH₂, NH₂, halogen, or cyano.

In second aspect of the present disclosure, there is provided a method for the preparation of the compound of Formula (I), its stereoisomers, intermediates, or pharmaceutically acceptable salts thereof, the method comprising reacting a compound of Formula II and a compound of Formula III in the presence of a base and a solvent; and incubating to form a compound of Formula (I),

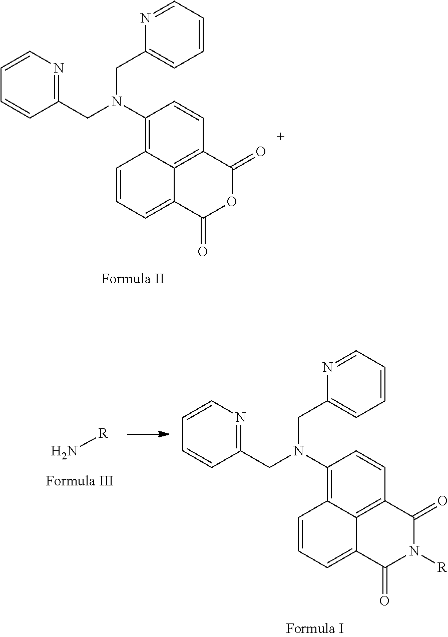

wherein R is C₁₋₁₀ alkyl substituted with a group selected from C₂₋₁₀ heterocyclyl, C₆₋₁₀ aryl, or C₂₋₁₀ heteroaryl, wherein C₂₋₁₀ heterocyclyl, C₆₋₁₀ aryl, or C₂₋₁₀ heteroaryl is optionally substituted with one or more substituents selected from hydrogen, hydroxyl, carboxyl, oxo, CONH₂, NH₂, C₁₋₁₀ alkyl, C₁₋₆ alkylamine, halogen, C₁₋₆ haloalkyl, cyano, or R¹,
R¹ is selected from C₁₋₁₀ alkyl, C₁₋₆ alkylamine, or C₁₋₆ haloalkyl, wherein C₁₋₁₀ alkyl, C₁₋₆ alkylamine, or C₁₋₆ haloalkyl is optionally substituted with C₆₋₁₀ aryl or C₂₋₁₀ heteroaryl, and wherein C₆₋₁₀ aryl or C₂₋₁₀ heteroaryl is optionally substituted with one or more substituents selected from hydroxyl, carboxyl, oxo, CONH₂, NH₂, halogen, or cyano.

In third aspect of the present disclosure, there is provided a pharmaceutical composition comprising the compound of Formula (I) and its stereoisomers, intermediates, or pharmaceutically acceptable salt thereof; a pharmaceutically acceptable carrier; and optionally, in combination with one or more other pharmaceutical compositions.

In fourth aspect of the present disclosure, there is provided a method for prevention or treatment of neurodegenerative disease, said method comprising administering to a subject an effective amount of the compound of Formula (I) and its stereoisomers, intermediates, or pharmaceutically acceptable salt thereof, or the pharmaceutical composition comprising the compound of Formula (I) and its stereoisomers, intermediates, or pharmaceutically acceptable salt thereof; a pharmaceutically acceptable carrier; and optionally, in combination with one or more other pharmaceutical compositions.

In fifth aspect of the present disclosure, there is provided a method for prevention or treatment of neurodegenerative disease, said method comprising administering a combination of the compound of Formula (I) and its stereoisomers, intermediates, or pharmaceutically acceptable salt thereof, or the pharmaceutical composition comprising the compound of Formula (I) and its stereoisomers, intermediates, or pharmaceutically acceptable salt thereof; a pharmaceutically acceptable carrier, with a clinically relevant immune modulator agent, to a subject in need of thereof.

In sixth aspect of the present disclosure, there is provided use of the compound of Formula (I) and its stereoisomers, intermediates, or pharmaceutically acceptable salt thereof or the pharmaceutical composition comprising the compound of Formula (I) and its stereoisomers, intermediates, or pharmaceutically acceptable salt thereof; a pharmaceutically acceptable carrier, for treatment of a condition mediated by aggregation of tau.

In seventh aspect of the present disclosure, there is provided use of the compound of Formula (I) and its stereoisomers, intermediates, or pharmaceutically acceptable salt thereof, or the pharmaceutical composition comprising the compound of Formula (I) and its stereoisomers, intermediates, or pharmaceutically acceptable salt thereof; a pharmaceutically acceptable carrier, with other clinically relevant agents or biological agents for treatment of a condition mediated by aggregation of tau.

These and other features, aspects, and advantages of the present subject matter will be better understood with reference to the following description. This summary is provided to introduce a selection of concepts in a simplified form. This summary is not intended to identify key features or essential features of the subject matter.

### BRIEF DESCRIPTION OF ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIG. 1 depicts A) Chemical grammar dictating LLPS (liquid-liquid phase separation) of macromolecules and rational design of small molecules by integrating chemical fragments with diverse chemical interactions to modulate LLPS of tau, B) Structure of rationally designed small molecules modulators, C) Schematic representation of modulation of Zn-mediated tau LLPS by designer small molecules, in accordance with an implementation of the present disclosure.

FIG. 2 depicts Zinc-small molecules interaction. A) Benesi-Hildebrand plots for interaction of small molecules 1a-c with Zn obtained from UV-Visible spectroscopic studies, B) Stern-Volmer plots of interaction of small molecules 1a-c with Zn obtained from steady state fluorescence measurements, C) and D) isothermal titration calorimetry (ITC) profiles of small molecule-Zn interaction for 1b and 1c respectively, in accordance with an implementation of the present disclosure.

FIG. 3 depicts small molecule-Tau interaction, A) Tau intrinsic fluorescence quenching with increasing concentration of small molecules shows their strong binding, B) Stern-Volmer plots of interaction of small molecules with tau protein obtained from steady state fluorescence measurements, C) Docked poses of small molecules with tau fibrils, D) Interactions of 1c with neighboring residues of proteins. Dashed lines represent hydrogen bonds while spikes depict hydrophobic interactions, in accordance with an implementation of the present disclosure.

FIG. 4 depicts DIC imaging of LLPS shows Zn induced tau LLPS A) inhibited by small molecules (Scale bar 10 μm), B) ability of small molecules to dissolve the phase separated condensates (Scale bar 10 μm), C) Analysis of droplet size revel that 1c effectively inhibit tau LLPS and D) reverse the phase separated condensates, E) UV-Visible absorbance spectra of 1c interaction with Zn, tau and mixture of both shows the stronger affinity towards tau, in accordance with an implementation of the present disclosure.

FIG. 5 depicts A) Biocompatibility of small molecules assessed by cell viability in (3-(4,5-dimethylthiazol-2-yl)-2,5-diphenyltetrazolium bromide) MTT assay, B) Fluorescent microscopy images of SHSY-5Y cells treated with 1a-c showed good cellular uptake and bioavailability, in accordance with an implementation of the present disclosure.

Figure S1 depicts A) UV-Visible absorbance spectral changes of 1a, 1b and 1c (20 μM) with increasing concentration of Zn (2.5 to 40 μM). B) Steady state fluorescent spectral changes of 1a, 1b and 1c (20 μM) with increasing concentration of Zn (2.5 to 40 μM), in accordance with an implementation of the present disclosure.

Figure S2 depicts ITC profiles of 1a-ZnCl₂ interaction, in accordance with an implementation of the present disclosure.

Figure S3 depicts fluorescent spectral changes of 1a, 1b and 1c (250 nM to 25 μM) upon interaction with tau protein (5 μM), in accordance with an implementation of the present disclosure.

Figure S4 depicts interactions of A) 1a and B) 1b with neighboring residues of tau protein. Dashed lines represent hydrogen bonds while spikes depict hydrophobic interactions, in accordance with an implementation of the present disclosure.

### DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

The term “at least one” used herein refers to one or more and thus includes individual components as well as mixtures/combinations.

The term “pharmaceutically acceptable salt” embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methane sulphonic, ethane sulphonic, benzene sulphonic or p-toluene sulphonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines.

Other preferred salts according to the invention are quaternary ammonium compounds wherein an equivalent of an anion (X−) is associated with the positive charge of the N atom. X− may be an anion of various mineral acids such as, for example, chloride, bromide, iodide, sulphate, nitrate, phosphate, or an anion of an organic acid such as, for example, acetate, maleate, fumarate, citrate, oxalate, succinate, tartrate, malate, mandelate, trifluoroacetate, methane sulphonate and p-toluene sulphonate. X− is preferably an anion selected from chloride, bromide, iodide, sulphate, nitrate, acetate, maleate, oxalate, succinate or trifluoroacetate. More preferably X− is chloride, bromide, trifluoroacetate or methane sulphonate. Nonlimiting examples of pharmaceutically acceptable salts include but are not limited to glycolate, fumarate, mesylate, cinnamate, isethionate, sulfate, phosphate, diphosphate, nitrate, hydrobromide, hydroiodide, succinate, formate, acetate, dichloroacetate, lactate, p-toluenesulfonate, pamitate, pidolate, pamoate, salicylate, 4-aminosalicylate, benzoate, 4-acetamido benzoate, glutamate, aspartate, glycolate, adipate, alginate, ascorbate, besylate, camphorate, camphorsulfonate, camsylate, caprate, caproate, cyclamate, laurylsulfate, edisylate, gentisate, galactarate, gluceptate, gluconate, glucuronate, oxoglutarate, hippurate, lactobionate, malonate, maleate, mandalate, napsylate, napadisylate, oxalate, oleate, sebacate, stearate, succinate, thiocyanate, undecylenate, and xinafoate.

The term “effective amount” means an amount of a compound or composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically-acceptable excipient(s)/carrier(s) utilized, the route of administration, and like factors within the knowledge and expertise of the attending physician.

Compounds of the present invention may be combined with a pharmaceutically acceptable carrier to provide pharmaceutical compositions useful for treating the conditions or disorders. The particular carrier employed in the pharmaceutical compositions may vary depending upon the type of administration desired (e.g. intravenous, oral, topical, suppository, or parenteral). For example, in preparing the compositions in oral liquid dosage forms (e.g. suspensions, elixirs and solutions), typical pharmaceutical media include but not limited to water, glycols, oils, alcohols, flavoring agents, preservatives, coloring agents and the like. Similarly, for preparing oral solid dosage forms (e.g. powders, tablets and capsules), carriers include but not limited to starches, sugars, diluents, granulating agents, lubricants, binders, disintegrating agents and the like.

Typical compositions include a compound of the invention and a pharmaceutically acceptable carrier. For example, the active compound will be mixed with a carrier, or diluted by a carrier, or enclosed within a carrier which can be in the form of an ampoule, capsule, sachet, paper, or other container. When the compound is mixed with a carrier, or when the carrier serves as a diluent, it can be solid, semi-solid, or liquid material that acts as a vehicle, excipient, or medium for the active compound. The compound can be adsorbed on a granular solid carrier, for example, contained in a sachet. Some examples of suitable carriers include but not limited to water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, agar, pectin, acacia, stearic acid or lower alkyl ethers of cellulose, silicic acid, fatty acids, fatty acid amines, fatty acid mono glycerides and diglycerides, pentaerythritol fatty acid esters, polyoxyethylene, hydroxymethylcellulose, and polyvinylpyrrolidone. Similarly, the carrier or diluent can include any sustained release material known in the art such as glyceryl monostearate or glyceryl distearate, alone or mixed with a wax.

The term “one or more other pharmaceutical composition” refers to other active pharmaceutical ingredients or composition that can work in combination with pharmaceutical composition of the present disclosure. The other pharmaceutical composition includes but not limited to Food and Drug Administration (FDA) approved drugs for the preliminary medication of AD patients and inflammation such as Aricept® (donepezil), Exelon® (rivastigmine), Razadyne® (galantamine), Namenda® (memantine), Nonsteroidal anti-inflammatory drugs (NSAIDs), Namzaric® (Donepezil and Memantine), Belsomra® (Suvorexant), and Aduhelm™ (Aducanumab).

In this specification, the prefix C_{x-y} as used in terms such as C_{x-y} alkyl and the like (where x and y are integers) indicates the numerical range of carbon atoms that are present in the group; for example, C₁₋₆ alkyl includes C₃ alkyl (propyl and isopropyl), C₄ alkyl (butyl, 1-methylpropyl, 2-methylpropyl, and t-butyl), and the like. Unless specifically stated, the bonding atom of a group may be any suitable atom of that group; for example, propyl includes prop-1-yl and prop-2-yl.

The term “C₁₋₁₀ alkyl” as used herein refers to a radical or group which may be saturated or unsaturated, linear or branched hydrocarbons, unsubstituted or mono- or poly-substituted. The term “alkyl” refers to a mono-radical, branched or unbranched, saturated hydrocarbon chain having from 1 to 10 carbon atoms. This term is exemplified by groups such as methyl, n-butyl, iso-butyl, t-butyl, n-hexyl, and the like. The groups may be optionally substituted.

The term “heteroaryl” refers to an aromatic cyclic group having 1, 2, 3, 4, 5, 6, 7, 8, 9, or 10, carbon atoms and 1, 2, 3 or 4 heteroatoms selected from oxygen, nitrogen and sulfur within at least one ring. Such heteroaryl groups can have a single ring (e.g. pyridyl or furyl) or multiple condensed rings (e.g. indolizinyl, benzothiazolyl, or benzothienyl). Examples of heteroaryls include, but are not limited to, tetrazole, [1,2,4] oxadiazole, [1,3,4] oxadiazole, [1,2,4] thiadiazole, [1,3,4] thiadiazole, pyrrole, imidazole, pyrazole, pyridine, pyrazine, pyrimidine, pyridazine, indolizine, isoindole, indole, indazole, purine, quinolizine, isoquinoline, quinoline, phthalazine, quinoxaline, quinazoline, cinnoline, pteridine, carbazole, carboline, phenanthridine, acridine, phenanthroline, isothiazole, phenazine, isoxazole, phenoxazine, phenothiazine, furan, thiophene, oxazole, thiazole, triazole, triazine and the like.

The term “heterocyclyl” refers to a saturated or partially unsaturated group having a single ring or multiple condensed rings, having from 2 to 10 carbon atoms and from 1 to 8 hetero atoms, preferably 1, 2, 3 or 4 heteroatoms, selected from nitrogen, sulfur, phosphorus, and/or oxygen within the ring. Heterocyclic groups can have a single ring or multiple condensed rings, and include tetrahydrofuranyl, morpholinyl, piperidinyl, piperazinyl, dihydropyridinyl, tetrahydroquinolinyl and the like. The groups may be optionally substituted.

The term “hydroxyl” refers to an —OH moiety attached to a main chain of carbon atoms.

The term “aryl” refers to any mono- and poly-carbocyclic ring systems wherein the individual carbocyclic rings in the polyring systems are fused or attached to each other via a single bond and wherein at least one ring is aromatic. Unless otherwise indicated, substituents to the aryl ring systems can be attached to any ring atom, such that the attachment results in the formation of a stable ring system. The aryl group may be optionally substituted.

The term “tau” refers to a class of microtubule-associated protein (MAP), helping to maintain and stabilize the microtubule assembly in matured neurons. Tau interacts with tubulin and stimulates its assembly into microtubules to maintain the structure and function of neuronal cells. The self-aggregation of hyperphosphorylated Tau form intracellular neurofibrillary tangles and paired helical filaments that is associated with the onset of neurodegenerative disorders like AD and taupathies.

The term “LLPS” refers to Liquid-liquid phase separation (LLPS) is a complex physicochemical phenomenon mediated by multivalent transient weak interactions among macromolecules like polymers, proteins, and nucleic acids. Liquid-liquid phase separation is a complex phenomenon that has role in cellular physiology and disease conditions like cancer and neurodegenerative disorders. The protein expression associated with cancer and the aggregation of proteins implicated in neurodegenerative disorders was regulated through LLPS.

The term “binding moiety of tau protein” refers to a chemical moiety that strongly binds to tau protein.

The term “NMI” refers to the naphthalene monoimide compounds and is denoted as NMIs or NMI compounds. The compounds of the present disclosure are compounds of NMIs and are referred to as “modulators aggregation of tau” or “modulators” or “multifunctional modulators”, “modulators of LLPS” as these compounds synergistically modulate various etiologies of AD. The compounds of the present disclosure are also referred to as “small molecules”.

A term once described, the same meaning applies for it, throughout the disclosure.

The compound of Formula (I), and its polymorphs, stereoisomers, prodrugs, solvates, co-crystals, intermediates, pharmaceutically acceptable salts, and metabolites thereof can also be referred as “compounds of the present disclosure” or “compounds”.

Furthermore, the compound of Formula (I), can be its stereoisomer's, or pharmaceutically acceptable salts and compositions. It is understood that included in the family of compounds of Formula (I), are isomeric forms including diastereoisomers, enantiomers, tautomers, and geometrical isomers in “E” or “Z” configurational isomer or a mixture of E and Z isomers. It is also understood that some isomeric forms such as diastereomers, enantiomers and geometrical isomers can be separated by physical and/or chemical methods by those skilled in the art.

Compounds disclosed herein may exist as single stereoisomers, racemates and or mixtures of enantiomers and/or diastereomers. All such single stereoisomers, racemates and mixtures thereof are intended to be within the scope of the subject matter described.

Compounds disclosed herein include isotopes of hydrogen, carbon, oxygen, fluorine, chlorine, iodine and sulfur which can be incorporated into the compounds, such as, but not limited to, ²H (D), ³H (T), ¹¹C, ¹³C, ¹⁴C, ¹⁵N, ¹⁸F, ³⁵S, ³⁶Cl, ⁶⁴Cu, and ¹²⁵I. Compounds of this disclosure wherein atoms were isotopically labeled for example radioisotopes such as ³H, ¹³C, ¹⁴C, and the like can be used in metabolic studies, kinetic studies, and imaging techniques such as positron emission tomography used in understanding the tissue distribution of the drugs. Compounds of the disclosure where hydrogen is replaced with deuterium may improve the metabolic stability, and pharmacokinetics properties of the drug such as in vivo half-life.

As discussed in the background, increasing number of dementia cases around the world is alarming to give immediate attention to developing better and more potent therapeutics for AD and other dementia cases. Although, several treatment options for the majority of cancer conditions, increasing resistance to therapeutics demand better therapeutics targeting drug targets are available, still the continuous efforts to develop therapeutics for AD, other tauopathies and dementia met with failures. There is a need to evolve novel targets that have major implications in disease development and progression. Recent studies have revealed critical role of LLPS in mediating the protein aggregation responsible for many neurodegenerative disorders. Accumulating evidence suggests LLPS as a tangible and novel target for AD, tauopathies, neurodegenerative disorders and cancer conditions. LLPS is a complex physicochemical phenomenon driven by transient, weak, multivalent interactions that is challenging to target by small molecules. Thus, the present disclosure has established a rational approach to integrate multiple chemical moieties to rationally design drug like small molecules to modulate tau LLPS. The designed small molecules are efficient in modulating LLPS, biocompatible and cell permeable. In fact, there are limited small molecules that has been demonstrated to have LLPS modulatory effect. The novel design strategy and small molecules of the present disclosure have potential for other protein like Aβ, alpha synuclein, FUS protein, TDP-43, IAPP, RNA-binding proteins (RBPs), HNRNPA1, TIA1, p53, etc., to develop therapeutics for neurodegenerative disorders and cancer conditions.

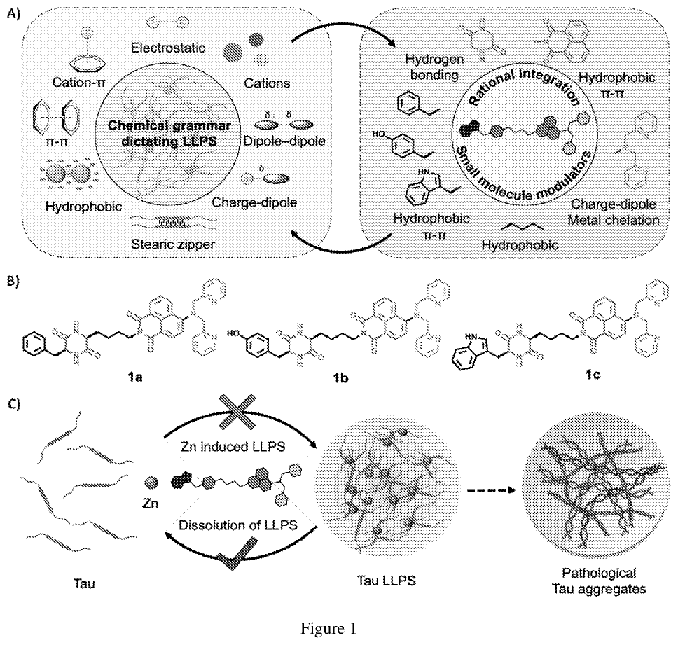

LLPS is a resultant of electro-chemical gradient force generated by the multivalent noncovalent interactions of biomolecules. The phase separated condensate is a dynamic membraneless structure continuously exchanging the biomolecules from surroundings, sensitive to external and internal stimuli that regulate their size and lifetime. Proteins with intrinsically disordered regions (IDRs) and RNAs undergo LLPS by intra- and inter-molecular interactions. The chemical grammar that governs the LLPS are hydrophobic contacts, steric zipper motifs, cations, charge-charge, charge-dipole, dipole-dipole, π-π, and cation-interactions (FIG. 1A) (Wang J et al A Molecular Grammar Governing the Driving Forces for Phase Separation of Prion-like RNA Binding Proteins. Cell. 2018; 174 (3): 688-699.e16 and Kilgore H R, Young R A, Learning the chemical grammar of biomolecular condensates. Nat. Chem. Biol. 2022. doi.10.1038/s41589-022-01046-y). Tau is a microtubule associated protein with IDRs that undergo LLPS and aggregation to form pathogenic species. Accumulation of intracellular tau tangles is evident in AD and many other tauopathies like progressive supranuclear palsy (PSP), corticobasal degeneration (CBD), argyrophilic grain disease, Pick's disease (PiD), frontotemporal dementia (FTD), and chronic traumatic encephalopathy (CTE). Tau being intrinsically disordered protein (IDP) with many charged amino acids composition and the absence of stable secondary structures results in many structural polymorphs enable it to engage in diverse multivalent interactions with itself and other biomolecules resulting in LLPS. Diverse interactions of tau are attributed to high percentage of proline, glycine, polar and charged amino acids content. Structurally, tau consist of two N-terminal inserts, proline rich domain, microtubule binding region (MTBR) with four repeat domains (4R) and a C-terminal domain. Tau phase separate into condensates by self-coacervation through homotypic interaction and complex coacervation by heterotypic interaction with other biomolecules such as RNA. The proline rich domain with potential phosphorylation sites, KXGS motifs of MTBR, Lys residues and stearic zipper motifs in repeat domains are the driving components of tau LLPS. The negatively charged N-terminal and positively charged middle/C terminal ends of tau induce electrostatic interaction that drive self-coacervation. Phase separation is also driven by hydrophobic interactions that promote the tau aggregation. Intracellular levels of zinc cation (Zn^{II}) is highly regulated due to its diverse functions. The altered Zn homeostasis is known to have a significant role in neuropathological conditions. Zinc binds to tau in a tetrahedral fashion through Cys-291 and Cys-322, His-330 and His-362 residues. It was reported that tau bear one high affinity (Kₐ=2.0±0.5×10⁶ M⁻¹) and three low affinity (Kₐ=5.9±1.7×10⁴ M⁻¹) Zn binding sites (Roman A Y, et al, Zinc Induces Temperature-Dependent Reversible Self-Assembly of Tau. J. Mol. Biol. 2019; 431 (4): 687-695). Recent NMR study showed three Zn binding sites on tau located in the N-terminal part (His-14, His-32, His-94, and His-121), MTBR (His-299, Cys-322, His-329 and His-330), and C-terminal part (His-362, His-374, His-388 and His-407) (La Rocca R et al., Identification of the three zinc-binding sites on tau protein. Int. J. Biol. Macromol. 2022; 209 (A): 779-784). Recent studies have demonstrated that Zn promotes tau LLPS in vitro, in cellular milieu and induces mitochondrial damage (Singh V, et al, Zinc promotes liquid-liquid phase separation of tau protein. J. Biol. Chem. 2020; 295 (18): 5850-5856).

The intracellular concentration of free tau is very low (nanomolar range), while high concentration (micromolar) drives its aggregation to form toxic species. LLPS increases the concentration of tau inside the liquid droplet that essentially drives the aggregation. In vitro and in vivo studies have demonstrated that tau undergoes dynamic LLPS to form gel-like structure leading to aggregation. The identification of toxic oligomers inside the condensate demonstrated the role of LLPS in driving tau monomers to toxic aggregates. Further, tau undergoes conformational changes inside the phase separated droplet that expose MTBR to promote aggregation. Majority of tau-targeted therapeutics (AD and tauopathies) include inhibitors of tau aggregation and immunotherapeutics directed at tau tangles. Accumulating evidence from the recent studies suggest LLPS is a critical step in tau aggregation and tangible drug target. There are limited chemical tools available to modulate tau LLPS due to complex chemical interactions driving the process. The underlying chemical grammar that dictates the complex process of LLPS offers insights for designing modulators by integrating suitable chemical moieties with diverse interactions (FIG. 1A). Development of small molecules capable of modulating complex multivalent and transient noncovalent interactions driven LLPS is a challenging task. Thus, the present disclosure attempted to rationally design small molecule by integrating different chemical moieties that are capable of interfering with the chemical grammar responsible for inducing LLPS process (FIG. 1B). The compounds of the present disclosure found to effectively inhibit Zn-mediated tau LLPS and dissolve the phase separated condensates (FIG. 1C). These drug-like molecules are biocompatible and cell permeable that underscore their potential for in vivo applications.

Unless defined otherwise, all technical and scientific terms used herein have the same meaning as commonly understood by one of ordinary skill in the art to which this disclosure belongs. Although any methods and materials similar or equivalent to those disclosed herein can be used in the practice or testing of the disclosure, the preferred methods, and materials are now described. All publications mentioned herein are incorporated herein by reference.

The present disclosure is not to be limited in scope by the specific embodiments disclosed herein, which are intended for the purposes of exemplification only. Functionally-equivalent products, compositions, and methods are clearly within the scope of the disclosure, as disclosed herein.

In an embodiment of the present disclosure, there is provided a compound of Formula (I)

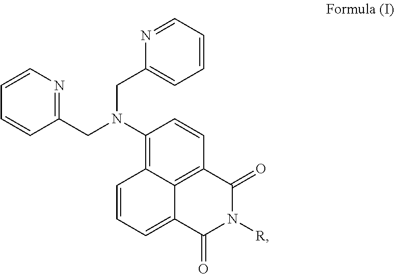

its stereoisomers, intermediates, or pharmaceutically acceptable salts thereof,
wherein
R is C₁₋₁₀ alkyl substituted with a group selected from C₂₋₁₀ heterocyclyl, C₆₋₁₀ aryl, or C₂₋₁₀ heteroaryl, wherein C₂₋₁₀ heterocyclyl, C₆₋₁₀ aryl, or C₂₋₁₀ heteroaryl is optionally substituted with one or more substituents selected from hydrogen, hydroxyl, carboxyl, oxo, CONH₂, NH₂, C₁₋₁₀ alkyl, C₁₋₆ alkylamine, halogen, C₁₋₆ haloalkyl, cyano, or R¹,
R¹ is selected from C₁₋₁₀ alkyl, C₁₋₆ alkylamine, or C₁₋₆ haloalkyl, wherein C₁₋₁₀ alkyl, C₁₋₆ alkylamine, or C₁₋₆ haloalkyl is optionally substituted with C₆₋₁₀ aryl or C₂₋₁₀ heteroaryl, and wherein C₆₋₁₀ aryl or C₂₋₁₀ heteroaryl is optionally substituted with one or more substituents selected from hydroxyl, carboxyl, oxo, CONH₂, NH₂, halogen, or cyano.

In an embodiment of the present disclosure, there is provided a compound of Formula (I), its stereoisomers, intermediates, or pharmaceutically acceptable salts thereof as disclosed herein, wherein R is C₁₋₁₀ alkyl substituted with C₂₋₁₀ heterocyclyl, wherein C₂₋₁₀ heterocyclyl is optionally substituted with one or more substituents selected from hydrogen, hydroxyl, carboxyl, oxo, CONH₂, NH₂, C₁₋₁₀ alkyl, C₁₋₆ alkylamine, halogen, C₁₋₆ haloalkyl, cyano or R¹,
R¹ is selected from C₁₋₁₀ alkyl, C₁₋₆ alkylamine, or C₁₋₆ haloalkyl, wherein C₁₋₁₀ alkyl, C₁₋₆ alkylamine, or C₁₋₆ haloalkyl is optionally substituted with C₆₋₁₀ aryl or C₂₋₁₀ heteroaryl, and wherein C₆₋₁₀ aryl or C₂₋₁₀ heteroaryl is optionally substituted with one or more substituents selected from hydroxyl, carboxyl, oxo, CONH₂, NH₂, halogen, or cyano.

In an embodiment of the present disclosure, there is provided a compound of Formula (I), its stereoisomers, intermediates, or pharmaceutically acceptable salts thereof as disclosed herein, wherein R is C₁₋₁₀ alkyl substituted with C₂₋₁₀ heterocyclyl, wherein C₂₋₁₀ heterocyclyl is substituted with one or more substituents selected from oxo, or R¹, R¹ is selected from C₁₋₁₀ alkyl, C₁₋₆ alkylamine, or C₁₋₆ haloalkyl, wherein C₁₋₁₀ alkyl, C₁₋₆ alkylamine, or C₁₋₆ haloalkyl is optionally substituted with C₆₋₁₀ aryl or C₂₋₁₀ heteroaryl, and wherein C₆₋₁₀ aryl or C₂₋₁₀ heteroaryl is optionally substituted with one or more substituents selected from hydroxyl, carboxyl, oxo, CONH₂, NH₂, halogen, or cyano.

In an embodiment of the present disclosure, there is provided a compound of Formula (I), its stereoisomers, intermediates, or pharmaceutically acceptable salts thereof as disclosed herein, wherein R is C₁₋₁₀ alkyl substituted with C₂₋₁₀ heterocyclyl, wherein C₂₋₁₀ heterocyclyl is substituted with one or more substituents selected from oxo or R¹, R¹ is C₁₋₁₀ alkyl substituted with C₆₋₁₀ aryl or C₂₋₁₀ heteroaryl, and wherein C₆₋₁₀ aryl or C₂₋₁₀ heteroaryl is optionally substituted with one or more substituents selected from hydroxyl, carboxyl, oxo, CONH₂, NH₂, halogen, or cyano.

In an embodiment of the present disclosure, there is provided a compound of Formula (I), its stereoisomers, intermediates, or pharmaceutically acceptable salts thereof as disclosed herein, wherein R is C₁₋₆ alkyl substituted with C₂₋₆ heterocyclyl, wherein C₂₋₆ heterocyclyl is substituted with one or more substituents selected from oxo or R¹, R¹ is selected from C₁₋₆ alkyl substituted with C₆₋₁₀ aryl, and wherein C₆₋₁₀ aryl is optionally substituted with hydroxyl.

In an embodiment of the present disclosure, there is provided a compound of Formula (I), its stereoisomers, intermediates, or pharmaceutically acceptable salts thereof as disclosed herein, wherein R is C₁₋₆ alkyl substituted with C₂₋₆ heterocyclyl, wherein C₂₋₆ heterocyclyl is substituted with one or more substituents selected from oxo or R¹, R¹ is C₁₋₆ alkyl substituted with C₂₋₁₀ heteroaryl.

In an embodiment of the present disclosure, there is provided a compound of Formula (I), its stereoisomers, intermediates, or pharmaceutically acceptable salts thereof as disclosed herein selected from (i) 2-(4-((2S,5S)-5-benzyl-3,6-dioxopiperazin-2-yl)butyl)-6-(bis(pyridin-2-ylmethyl)amino)-1H-benzo[de]isoquinoline-1,3(2H)-dione of Formula (Ia); (ii) 6-(bis(pyridin-2-ylmethyl)amino)-2-(4-((2S,5S)-5-(4-hydroxybenzyl)-3,6-dioxopiperazin-2-yl)butyl)-1H-benzo[de]isoquinoline-1,3(2H)-dione of Formula (Ib); and (iii) 2-(4-((2S,5S)-5-((1H-indol-3-yl)methyl)-3,6-dioxopiperazin-2-yl)butyl)-6-(bis(pyridin-2-ylmethyl)amino)-1H-benzo[de]isoquinoline-1,3(2H)-dione of Formula (Ic).

In an embodiment of the present disclosure, there is provided a compound of Formula (I), its stereoisomers, intermediates, or pharmaceutically acceptable salts thereof as disclosed herein, for use in the manufacture of a medicament for treating a neurodegenerative disease selected from Alzheimer's disease (AD), Pick's disease, tauopathies, frontotemporal dementia associated with tau-immunoreactive inclusions (FTD-tau), progressive supranuclear palsy (PSP), corticobasal degeneration (CBD), chronic traumatic encephalopathy, primary age related tauopathy, argyrophilic grain disease, or post-encephalitic parkinsonism.

In an embodiment of the present disclosure, there is provided a compound of Formula (I), its stereoisomers, intermediates, or pharmaceutically acceptable salts thereof as disclosed herein, wherein the compound modulates aggregation of tau.

In an embodiment of the present disclosure, there is provided a compound of Formula (I), its stereoisomers, intermediates, or pharmaceutically acceptable salts thereof as disclosed herein, wherein the compound is capable of binding with Zn²⁺ ion and tau protein.

In an embodiment of the present disclosure, there is provided a compound of Formula (I), its stereoisomers, intermediates, or pharmaceutically acceptable salts thereof as disclosed herein, wherein the compound modulates tau liquid-liquid phase separation.

In an embodiment of the present disclosure, there is provided a compound of Formula (I), its stereoisomers, intermediates, or pharmaceutically acceptable salts thereof as disclosed herein, wherein the compound is capable of inhibiting or dissolution of Zinc mediated liquid-liquid phase separated condensate and aggregates of tau.

In an embodiment of the present disclosure, there is provided a chelate complex comprising a chelating ligand comprising the compound of Formula (I), its stereoisomers, intermediates, or pharmaceutically acceptable salts thereof as disclosed herein, with one or more metal ions; and, optionally, a binding moiety of tau protein.

In an embodiment of the present disclosure, there is provided a chelate complex comprising a chelating ligand comprising the compound of Formula (I), its stereoisomers, intermediates, or pharmaceutically acceptable salts thereof as disclosed herein, with Zn²⁺ as metal ions; and, optionally, a binding moiety of tau protein.

In an embodiment of the present disclosure, there is provided a method for the preparation of the compound of Formula (I), its stereoisomers, intermediates, or pharmaceutically acceptable salts thereof as disclosed herein, the method comprising reacting a compound of Formula II and a compound of Formula III in presence of a base and a solvent; and incubating to form a compound of Formula (I)

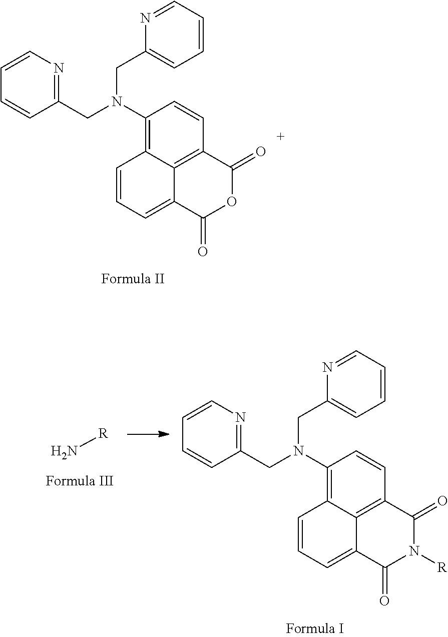

wherein
R is C₁₋₁₀ alkyl substituted with a group selected from C₂₋₁₀ heterocyclyl, C₆₋₁₀ aryl, or C₂₋₁₀ heteroaryl, wherein C₂₋₁₀ heterocyclyl, C₆₋₁₀ aryl, or C₂₋₁₀ heteroaryl is optionally substituted with one or more substituents selected from hydrogen, hydroxyl, carboxyl, oxo, CONH₂, NH₂, C₁₋₁₀ alkyl, C₁₋₆ alkylamine, halogen, C₁₋₆ haloalkyl, cyano, or R¹,
R¹ is selected from C₁₋₁₀ alkyl, C₁₋₆ alkylamine, or C₁₋₆ haloalkyl, wherein C₁₋₁₀ alkyl, C₁₋₆ alkylamine, or C₁₋₆ haloalkyl is optionally substituted with C₆₋₁₀ aryl or C₂₋₁₀ heteroaryl, and wherein C₆₋₁₀ aryl or C₂₋₁₀ heteroaryl is optionally substituted with one or more substituents selected from hydroxyl, carboxyl, oxo, CONH₂, NH₂, halogen, or cyano.

In an embodiment of the present disclosure, there is provided a method for the preparation of the compound of Formula (I), its stereoisomers, intermediates, or pharmaceutically acceptable salts thereof as disclosed herein, wherein said incubation is performed at a temperature in a range of 100 to 120 degree Celsius for a period of 4 to 16 hours.

In an embodiment of the present disclosure, there is provided a method for the preparation of the compound of Formula (I), its stereoisomers, intermediates, or pharmaceutically acceptable salts thereof as disclosed herein, wherein the base is selected from triethylamine, diisopropylethylamine, 1,8-diazabicyclo(5.4.0) undec-7-ene, pyridine, or combinations thereof; and the solvent is selected from dimethylformamide, dimethylsulphoxide, isopropyl alcohol, or combinations thereof.

In an embodiment of the present disclosure, there is provided a method for the preparation of the compound of Formula (I), its stereoisomers, intermediates, or pharmaceutically acceptable salts thereof as disclosed herein, wherein the method comprises purifying the compound of Formula (I), by centrifuging to obtain a precipitate of the compound of Formula I and washing the precipitate.

In an embodiment of the present disclosure, there is a pharmaceutical composition comprising the compound of Formula (I) its stereoisomers, intermediates, or pharmaceutically acceptable salts thereof as disclosed herein, with a pharmaceutically acceptable carrier, optionally in combination with one or more other pharmaceutical compositions.

In an embodiment of the present disclosure, there is a pharmaceutical composition comprising the compound of Formula (I), its stereoisomers, intermediates, or pharmaceutically acceptable salts thereof as disclosed herein, wherein the composition is in a form selected from tablet, capsule, powder, syrup, solution, aerosol, or suspension.

In an embodiment of the present disclosure, there is provided a method for prevention or treatment of a condition mediated by a neurodegenerative disease, said method comprising administering to a subject an effective amount of the compound of Formula (I), its stereoisomers, intermediates, or pharmaceutically acceptable salts thereof as disclosed herein.

In an embodiment of the present disclosure, there is provided a method for prevention or treatment of a condition mediated by a neurodegenerative disease, said method comprising administering to a subject an effective amount of the pharmaceutical composition as disclosed herein.

In an embodiment of the present disclosure, there is provided a method of treatment of a condition mediated by a neurodegenerative disease, said method comprising administering a combination of the compound of Formula (I) its stereoisomers, intermediates, or pharmaceutically acceptable salts thereof as disclosed herein with other clinically relevant immune modulator agents to a subject in need of thereof.

In an embodiment of the present disclosure, there is provided a method of treatment of a condition mediated by a neurodegenerative disease, said method comprising administering a combination of the pharmaceutical composition as disclosed herein with other clinically relevant immune modulator agents to a subject in need of thereof.

In an embodiment of the present disclosure, there is provided a method for the treatment of a condition mediated by the disorder selected from Alzheimer's disease (AD), Pick's disease, tauopathies, frontotemporal dementia associated with tau-immunoreactive inclusions (FTD-tau), progressive supranuclear palsy (PSP), corticobasal degeneration (CBD), chronic traumatic encephalopathy, primary age related tauopathy, argyrophilic grain disease, or post-encephalitic parkinsonism.

In an embodiment of the present disclosure, there is provided use of the compound of Formula (I) its stereoisomers, intermediates, or pharmaceutically acceptable salts thereof as disclosed herein for treatment of a condition mediated by aggregation of tau.

In an embodiment of the present disclosure, there is provided use of the pharmaceutical composition as disclosed herein for the treatment of a condition mediated by aggregation of tau.

Although the subject matter has been described in considerable detail with reference to certain examples and implementations thereof, other implementations are possible.

### EXAMPLES

The disclosure will now be illustrated with the working examples, which is intended to illustrate the working of disclosure and not intended to take restrictively to imply any limitations on the scope of the present disclosure. Unless defined otherwise, all technical and scientific terms used herein have the same meaning as commonly understood to one ordinary person skilled in the art to which this disclosure belongs. Although methods and materials similar or equivalent to those disclosed herein can be used in the practice of the disclosed methods and compositions, the exemplary methods, devices and materials are disclosed herein. It is to be understood that this disclosure is not limited to particular methods, and experimental conditions described, as such methods and conditions may apply.

### Materials and Methods

All the chemicals and solvents were purchased from Merck or Spectrochem and used without further purification unless mentioned. Reagents for protein expression and purification were purchased from HiMedia or Invitrogen and used as per the manufacturer's instructions. Ni-NTA (nickel-nitrilotriacetic acid) resins were purchased from Invitrogen and used as per the manufacturer protocols. Antibiotics, Luria-Bertani (LB) powder, isopropyl-β-D-thiogalactoside (IPTG) were purchased from HiMedia. Acrylamide, bis-acrylamide, ammonium persulfate (APS), tetramethylethylenediamine (TEMED), 3-(4,5-dimethylthiazol-2-yl)-2,5-diphenyltetrazolium bromide (MTT), ZnCl₂, polyethylene glycol-4000 (PEG-4000) and (4-(2-hydroxyethyl)-1-piperazineethanesulfonic acid) (HEPES) and phenylmethanesulfonyl fluoride (PMSF) were purchased from Merck. Broadford reagents and Coomassie brilliant blue (CBB), SDS-PAGE sample loading buffer and protein ladder were purchased from BioRad. Dulbecco's modified eagle medium F-12 (DMEM F12), fetal bovine serum (FBS) and penicillin-streptomycin (PS) were purchased from Gibco, Invitrogen. All sterile plastic wares for cell culture were purchased from Nunc, ThermoFisher Scientific. 1H NMR were recorded on Bruker AV-400 (400 MHz) spectrometer with TMS (tetramethylsilane) as internal standard. 13C NMR spectra were recorded on Bruker AV-400 (101 MHz) and Jeol-600 (150 MHz) spectrophotometers. High-resolution mass spectra (HRMS) were obtained from Agilent 6538 UHD HRMS/Q-TOF high-resolution spectrometer. The absorption spectra were recorded on Agilent Cary Eclipse Series UV-Vis-NIR spectrophotometer and fluorescence spectra were recorded on Agilent Cary Eclipse Fluorescence Spectrophotometer. The absorbance in well plates were recorded in Spectramax i3 (Molecular devices) plate reader. The isothermal titration calorimetry (ITC) measurements were performed in Malvern MicroCal-PEAQ ITC instrument. The microscopy imaging was performed in Olympus Fluoview-3000 confocal laser scanning microscope and Leica DMi8 fluorescent microscope with live cell imaging set up and analysed by ImageJ software. All the plotting and data analysis was performed in Origin (Pro), Version 2022 and GraphPad Prism 9.4.0 software.

### Example 1

### Synthesis of Compounds of Formula (I)

The compounds of Formula (I) of the present disclosure were synthesized wherein R is C₁₋₁₀ alkyl substituted with a group selected from C₂₋₁₀ heterocyclyl, C₆₋₁₀ aryl, or C₂₋₁₀ heteroaryl, wherein C₂₋₁₀ heterocyclyl, C₆₋₁₀ aryl, or C₂₋₁₀ heteroaryl is optionally substituted with one or more substituents selected from hydrogen, hydroxyl, carboxyl, oxo, CONH₂, NH₂, C₁₋₁₀ alkyl, C₁₋₆ alkylamine, halogen, C₁₋₆ haloalkyl, cyano, or R¹,
R¹ is selected from C₁₋₁₀ alkyl, C₁₋₆ alkylamine, or C₁₋₆ haloalkyl, wherein C₁₋₁₀ alkyl, C₁₋₆ alkylamine, or C₁₋₆ haloalkyl is optionally substituted with C₆₋₁₀ aryl or C₂₋₁₀ heteroaryl, and wherein C₆₋₁₀ aryl or C₂₋₁₀ heteroaryl is optionally substituted with one or more substituents selected from hydroxyl, carboxyl, oxo, CONH₂, NH₂, halogen, or cyano. Pharmaceutically acceptable salts of the compounds may be obtained as per procedures reported in the literature. Scheme 1 represents the general scheme of preparation of compounds of the present disclosure. Scheme 2 represents the synthetic preparation of different compounds from the parent naphthalene monoanhydride molecule (compound 1a-c).

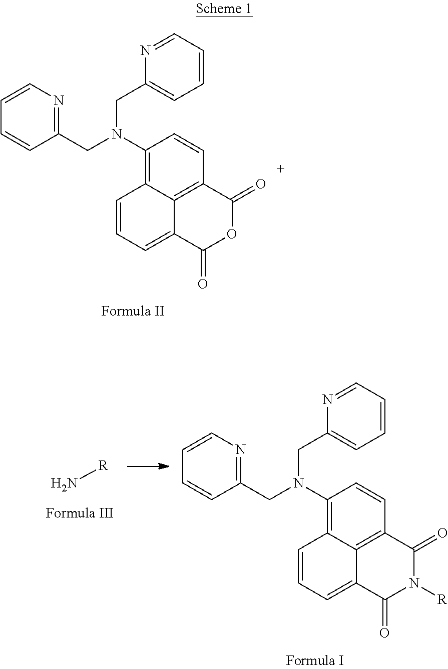

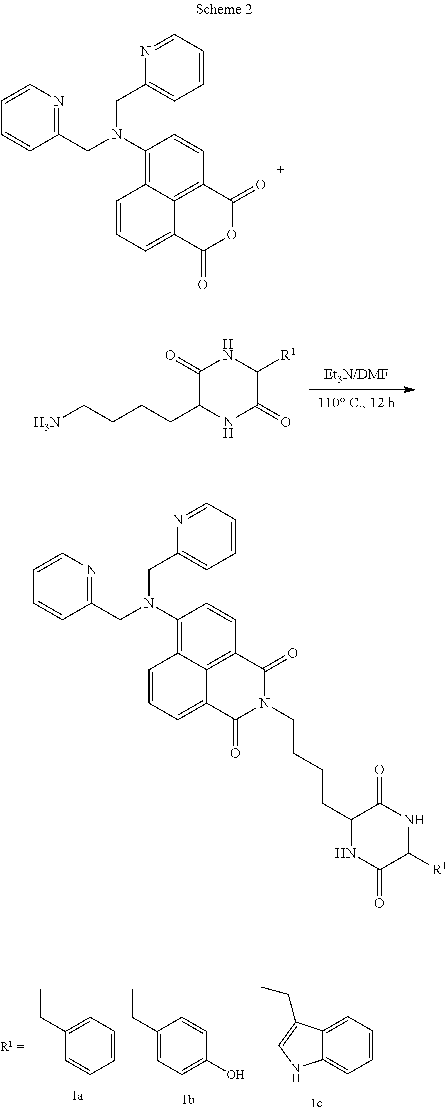

The compounds of the present disclosure were prepared by Scheme 2 as depicted above. The cyclic dipeptides were synthesized by following previously reported procedure (ACS Chem. Neurosci. 2022; 13 (14): 2209-2221 and J. Org. Chem. 2020; 85 (3): 1525-1536). 4-dipicolylamino-1,8-naphthalic anhydride (1 equiv, 75 mg, 0.189 mmol) and cyclo(Lys-Phe) (1.5 equiv, 80 mg, 0.284 mmol) were taken in a round bottom flask. To this mixture dimethyl formamide (DMF) (1.0 mL) and triethylamine (4.0 equiv, 106 μL, 0.76 mmol) were added. The resultant suspension was stirred at 110° C. for overnight. Then, to the reaction mixture, water (15 mL) was added to precipitate the desired product and to remove the remained compound. The resultant suspension was centrifuged to obtain the precipitate. The obtained precipitate was washed with water (15 mL). Finally, the obtained small molecules were purified by silica gel column chromatography using dichloromethane and methanol as eluents.

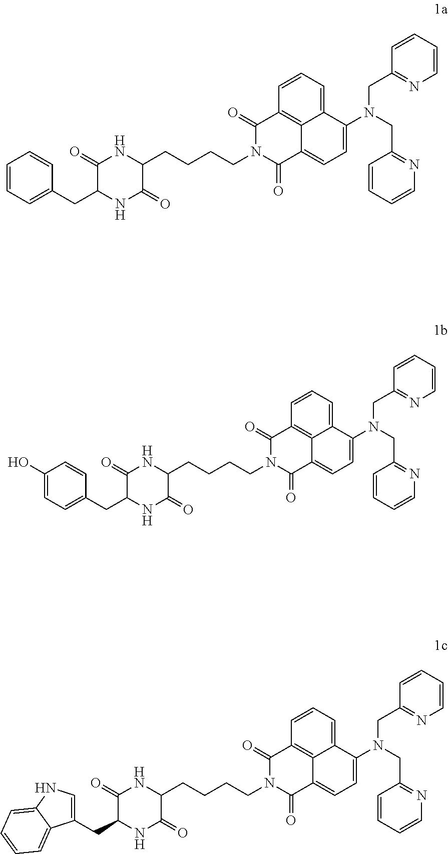 [SMILES: O=C1NC(Cc2ccccc2)C(=O)NC1CCCCN1C(=O)c2cccc3c(N(Cc4ccccn4)Cc4ccccn4)ccc(c23)C1=O ; O=C1NC(Cc2ccc(O)cc2)C(=O)NC1CCCCN1C(=O)c2cccc3c(N(Cc4ccccn4)Cc4ccccn4)ccc(c23)C1=O ; O=C1N[C@@H](Cc2c[nH]c3ccccc23)C(=O)NC1CCCCN1C(=O)c2cccc3c(N(Cc4ccccn4)Cc4ccccn4)ccc(c23)C1=O]

### Synthesis of Compounds 1a, 1b and 1c.

1a: The pure product was obtained as dark yellow solid (80 mg, 65%). ¹H NMR (400 MHz, DMSO-d₆): δ 8.95 (d, J=8.4 Hz, 1H), 8.54 (d, J=4.0 Hz, 2H), 8.48 (d, J=7.1 Hz, 1H), 8.24 (d, J=8.1 Hz, 1H), 8.12 (s, 1H), 8.03 (s, 1H), 7.81 (t, J=7.9 Hz, 1H), 7.72 (t, J=7.3 Hz, 2H), 7.46 (d, J=7.7 Hz, 2H), 7.37-7.06 (m, 8H), 4.72 (s, 4H), 4.17 (s, 1H), 3.92-3.74 (m, 2H), 3.60 (s, 1H), 3.13 (dd, J=13.2, 3.2 Hz, 1H), 2.84 (dd, J=13.3, 4.4 Hz, 1H), 1.31-1.23 (m, 2H), 1.11-1.07 (m, 1H), 0.85-0.76 (m, ³H). 13C NMR (100 MHz, DMSO-d₆): δ 166.9, 166.1, 163.4, 162.8, 157.3, 153.8, 149.2, 136.8, 136.1, 131.4, 130.6, 130.3, 129.4, 127.9, 126.7, 126.0, 122.6, 122.5, 122.3, 117.1, 115.2, 59.1, 55.3, 53.8, 40.1, 38.1, 33.0, 27.2, 21.1. HRMS (ESI-TOF) m/z: [M+H]⁺ Calcd for C₃₉H₃₆N₆O₄, 653.2871; Found, 653.2869.

1b: The pure product was obtained as green solid (100 mg, 79%). ¹H NMR (400 MHz, DMSO-d₆): δ 9.11 (s, 1H), 8.95 (d, J=8.5 Hz, 1H), 8.54 (d, J=4.6 Hz, 2H), 8.47 (d, J=7.2 Hz, 1H), 8.24 (d, J=8.2 Hz, 1H), 8.03 (d, J=10.9 Hz, 2H), 7.81 (t, J=7.9 Hz, 1H), 7.72 (td, J=7.7, 1.5 Hz, 2H), 7.45 (d, J=7.8 Hz, 2H), 7.32-7.22 (m, 3H), 6.94 (d, J=8.3 Hz, 2H), 6.63 (d, J=8.3 Hz, 2H), 4.72 (s, 4H), 4.08 (s, 1H), 3.94-3.79 (m, 2H), 3.60 (s, 1H), 3.01 (dd, J=13.6, 3.8 Hz, 1H), 2.73 (dd, J=11.6, 6.7 Hz, 1H), 1.42-1.29 (m, 2H), 1.19 (t, J=13.6 Hz, 1H). ¹³C NMR (100 MHz, DMSO-d₆): δ 166.9, 166.3, 163.4, 162.8, 157.3, 156.2, 153.8, 149.2, 149.1, 136.8, 131.4, 131.2, 130.6, 129.4, 126.04, 125.9, 122.6, 122.5, 122.3, 117.1, 115.2, 114.8, 59.1, 55.5, 53.8, 40.1, 37.4, 33.1, 27.4, 21.3. HRMS (ESI-TOF) m/z: [M+H]⁺ Calcd for C₃₉H₃₆N₆O₅, 669.2820; Found, 669.2819.

1c: The pure product was obtained as light brown solid (71 mg, 54%). ¹H NMR (400 MHz, DMSO-d₆) δ 10.86 (s, 1H), 8.94 (d, J=8.5 Hz, 1H), 8.54 (d, J=4.3 Hz, 2H), 8.47 (d, J=7.0 Hz, 1H), 8.24 (d, J=8.2 Hz, 1H), 8.02 (s, 1H), 7.95 (s, 1H), 7.85-7.77 (m, 1H), 7.73 (td, J=7.7, 1.6 Hz, 2H), 7.56 (d, J=7.8 Hz, 1H), 7.46 (d, J=7.8 Hz, 2H), 7.33-7.20 (m, 4H), 7.05 (d, J=2.1 Hz, 1H), 6.95 (t, J=7.2 Hz, 1H), 6.88 (t, J=7.3 Hz, 1H), 4.72 (s, 4H), 4.10 (s, 1H), 3.74 (t, J=7.4 Hz, 2H), 3.55 (s, 1H), 3.24 (dd, J=14.4, 4.1 Hz, 1H), 3.01 (dd, J=14.4, 4.5 Hz, 1H), 1.29-1.13 (m, 2H), 1.13-0.98 (m, 1H), 0.80-0.51 (m, 3H). ¹³C NMR (100 MHz, DMSO-d₆) δ 167.1, 166.9, 163.4, 162.8, 157.2, 153.7, 149.2, 136.9, 135.9, 131.4, 130.6, 129.4, 127.8, 126.0, 124.6, 122.6, 122.5, 122.4, 120.7, 118.9, 118.2, 117.2, 115.2, 111.1, 108.6, 59.1, 55.3, 53.9, 40.1, 33.1, 28.9, 27.3, 21.0. HRMS (ESI-TOF) m/z: [M+H]⁺ Calcd for C₄₁H₃₇N₇O₄, 692.2980; Found, 692.2986.

In another example, a pharmaceutical composition was prepared using the any of the compound of Formula I (Ia, Ib, and Ic) along with a pharmaceutical carrier such as one or more pharmaceutical media (solvents), flavoring agents, preservatives, coloring agents, starches, sugars, diluents, granulating agents, lubricants, binders, disintegrating agents or combinations thereof, for treating the conditions or disorders.

### Example 2

### Example 2.1: Expression and Purification of Tau Protein

Tau protein was expressed and purified from E. coli as per known procedures. Briefly, the full-length tau plasmid construct was transformed into E. coli, BL21 strain and cultured in LB broth containing ampicillin (100 μM) and chloramphenicol (50 μM) at 37° C. with continuous shaking at 180 rpm overnight. Then 1% of primary culture was transferred to 1 L of LB broth with antibiotics and incubated until it reaches OD of 0.6. The culture was induced with 1 mM IPTG and further cultured for 4 h. Cells were pelleted by centrifugation (7000 rpm) at 4° C. and resuspended in 50 mM phosphate buffer (0.3 M NaCl, 5 mM imidazole and 1 mM of PMSF, pH 8) and boiled to precipitate unwanted proteins for 10-15 min with intermittent agitation. The supernatant containing tau protein was collected and further purified by Ni-NTA affinity column chromatography. The purified tau protein was quantified by Bradford assay, characterised by SDS-PAGE and stored at −80° C. until further use.

### Example 2.2: UV-Visible Absorption and Fluorescence Spectroscopy

For UV-Visible absorbance spectroscopy, the compounds of 20 mM stock solution were prepared in DMSO and used for further experiments. The measurements were conducted in HEPES buffer (10 mM, pH 7.4) and phosphate buffered saline (PBS, 10 mM, pH 7.4) at room temperature. The data was plotted and analysed in Origin (Pro), Version 2022 software.

### Example 2.3: Metal Binding Experiments

The metal chelation ability of the compounds were assessed by UV-Visible absorbance and fluorescence spectroscopy by metal titration experiments. The small molecules were dissolved in HEPES buffer to make concentration of 20 μM and the metal ion (Zn²⁺) was titrated with increasing concentration from 2.5 μM to 40 μM with 2 min of incubation between each addition. The change in absorbance and fluorescence spectra of compound was recorded and analysed for metal-small molecules interaction. The data was plotted in Origin (Pro), Version 2022 and analysed for determination of dissociation constant. The absorbance values were fitted using Benesi-Hildebrand equation to elucidate the binding constant of metals with small molecules.

$$\frac{1}{\vert {A}_{0} - A \vert} = \frac{1}{\vert {A}_{α} - A \vert} = \frac{1}{\vert {A}_{α} - A \vert {K}_{B} [ Q ]}$$

where, K_{B} is the binding or association constant, and A₀ and A_{α} represent the peak absorbance of small molecules in the absence and presence of the highest concentration of metal ion (40 μM). The reciprocal of K_{B} was taken as the dissociation constant, K_{d}. The change in fluorescence was fitted using Stern-Volmer equation to determine K_{SV}:

$$\frac{{F}_{0}}{F} = 1 + {K}_{SV} [ Q ]$$

where, F₀ and F are the fluorescence intensities of small molecules in the absence and presence of metal ion, respectively. K_{SV} is the Stern-Volmer quenching constant and [Q] is the concentration of quencher/enhancer (metal ion in this case).

### Example 2.4: ITC Measurements

ITC measurements were performed using a Malvern MicroCal-PEAQ ITC machine. Solutions of small molecules (compounds 1a, 1b and 1c) of the present disclosure and ZnCl₂ were prepared in 100% DMSO. A revolving syringe was used to inject 26 consecutive 1.5 μL aliquots of Zn²⁺ into the small molecules solution to ensure a continual mixing. The ITC cell reference power and temperature were set at 5 cal/s and 25° C., respectively. To guarantee complete equilibration before the next injection, an interval of 150 s was maintained between subsequent measurements. Obtained isotherms were corrected by the subtraction of heat of dilution (buffer and small molecules). By using a single site binding model in the MicroCal PEAQ-ITC analysis software, nonlinear least squares fitting of the binding isotherms was carried out to estimate the thermodynamic parameters and dissociation constant (K_{d}).

### Example 2.5: Intrinsic Fluorescence Quenching Assay

The tau protein exhibits intrinsic fluorescence and the interaction of molecules with tau disturbed this and resulted in fluorescence quenching, which was monitored to understand the small molecules interaction with tau protein. Tau was dissolved in PBS buffer pH 7.4 to make 5 μM and titrated with small molecules from 250 nM to 25 μM concentration and fluorescence spectra was recorded by exiting at 274 nm. The emission spectra were plotted and analysed using Origin (Pro), Version 2022 and GraphPad Prism 9.4.0, fitted with one site binding saturation equation to find K_{d}. Further fluorescence quenching was fitted with Stern-Volmer equation to determine K_{SV}.

### Example 2.6: Microscopy Imaging

Liquid-liquid phase separation (LLPS) of tau was induced by molecular crowding in LLPS buffer (10 mM HEPES, 100 mM NaCl, 10% PEG 4000 and pH 7.4) and Zn.³² Tau (10 μM) was suspended in LLPS buffer and added Zn (40 μM) incubate for 10 min to induce LLPS. For inhibition studies the tau in LLPS buffer was treated with small molecules (20 μM) before addition of Zn and for control same volume of vehicle (DMSO) was added. Dissolution samples were prepared by induction of LLPS for 10 min using Zn and followed by addition of small molecules and further incubation of 15 min. The samples were drop-cast (10 μL) on clean glass slides, mount with coverslip and immediately imaged in Olympus FLUOVIEW 3000 microscope in differential interface contrast (DIC) mode with 63× oil immersion objective. The images were acquired in multiple areas and planes to confirm the LLPS formation and its modulation by small molecules. Image analysis was performed by using ImageJ software, plotted normalised droplet size against control and analysed by GraphPad Prism.

### Example 2.7: Cell Culture, MTT Assay and Cell Permeability Studies

Neuroblastoma cells (SHSY-5Y) were cultured in DMEM-F12 media supplemented with 10% FBS and 1% PS incubated in humidified chamber with 5% CO₂ maintained at 37° C. temperature. For MTT assay the cells were seeded in 96 well plate at the density of 20000 cells per well, different concentration of compounds treated and incubated. After 24 h cells were treated with 10 μL of MTT solution (5 mg/mL) and incubated for 3 h to form formazan crystals. The crystals were dissolved in DMSO:methanol (1:1) solution and absorbance was recorded at 570 nm wavelength and calculated percentage cell viability normalised to control (vehicle DMSO treatment). For cell permeability studies of small molecules, SHSY-5Y cells were cultured in confocal dishes, after 24 h treated with small molecule (10 μM) for 1 h. Then the cells were imaged in fluorescence microscope in green channel to visualise the cellular uptake and localisation of small molecules.

### Example 3

Interaction of Small Molecules with Zn²⁺

Compounds 1a-c are equipped with dipyridyl amine (DPA) moiety to chelate and inhibit Zn²⁺ mediated tau LLPS. The zinc binding ability of the compounds was evaluated by UV-Visible absorption spectroscopy, steady state fluorescence spectroscopy and isothermal titration calorimetry (ITC).

### Example 3.1: UV-Visible Absorption Spectroscopy

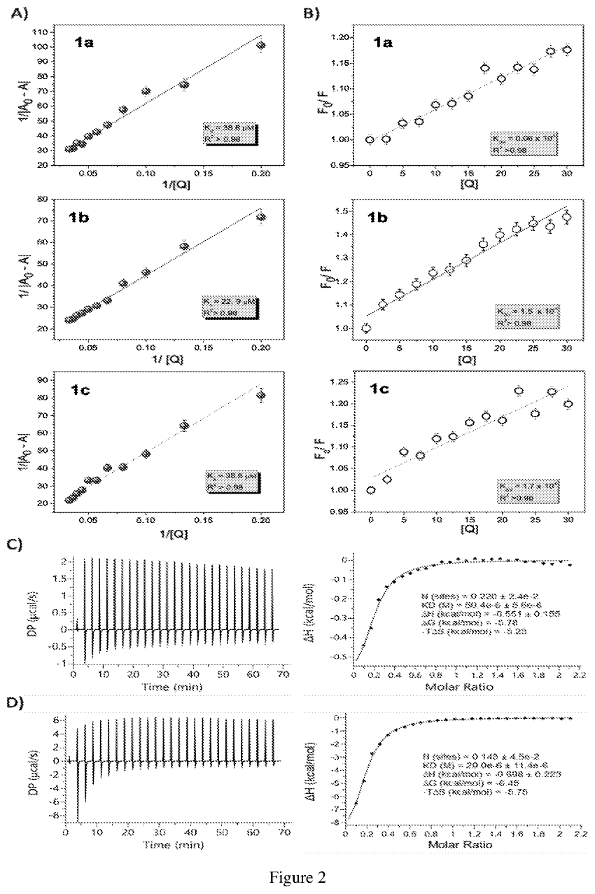

The absorption spectra of compounds showed hypochromic shifts upon the addition of increasing concentration of Zn²⁺ (2.5 to 40 μM). The absorption bands at ˜280 nm and ˜420 nm diminished with maximum reduction in the absorbance was observed for 1c, followed by 1b and 1c (Figure S1A). To quantitatively evaluate the binding affinity, the absorbance data was fitted with the Benesi-Hildebrand equation, which allowed to determine the binding constant (K_{B}). Linear fitting was obtained in all cases and the reciprocal of K_{B} was taken as the dissociation constant (K_{d}), which is a measure of the propensity of a complex to dissociate into its individual components. Compounds 1a-c exhibited K_{d} in the micromolar range, implying a strong interaction with Zn²⁺ (FIG. 2A). Compound 1c showed superior K_{d} value of 20.2 μM, followed by 1b (K_{d}=22.9 μM), while 1a exhibited relatively weak binding with K_{d} of 38.8 μM. Notably, the K_{d} values of all three compounds are in similar range, implying the negligible influence of amino acid moieties of CDP on metal chelation ability.

### Example 3.2: Fluorescence Spectroscopy

Fluorescence spectroscopy is a reliable method to probe host-probe interactions, wherein the change in wavelength or intensity of emission spectrum is indicative of the strength of complexation. Quenching of the fluorescence intensity was observed in case of all small molecules upon addition of Zn²⁺. The classical Stern-Volmer equation was used to quantify the quenching and measure the Stern-Volmer constant, K_{SV} wherein a higher value of K_{SV} implies ability of the compound to form strong complexation with the host (Zn²⁺). Compounds 1a-c showed prominent emission bands at 530 nm which decreases on addition of Zn²⁺ in a concentration dependent manner (Figure S1B). The obtained K_{SV} in case of all small molecules corroborates the fact that the interaction of metal ions with each of the compounds is robust, while being comparable and thus are in good agreement with the inferences obtained from absorbance measurements. Compound 1c showed highest K_{SV} (1.7×10⁴) followed by 1b (K_{SV}=1.7×10⁴) and 1a (0.06×10⁴) (FIG. 2B). The tau-Zn interactions have been characterized earlier and found that it has one high affinity binding site and three low affinity binding sites. Due to the good binding affinity of the small molecules towards Zn, the designed small molecules were anticipated to chelate and interfere tau-Zn interaction.

### Example 4: ITC Study of Small Molecules-Zn Binding Interaction

The thermodynamic parameters of the interactions between small molecules and Zn²⁺ were determined by ITC study. ZnCl₂ solution in the injection syringe was titrated into compound solution in the sample cell. The heat change was measured and plotted as a function of the molar ratio of small molecules and Zn²⁺. All relevant thermodynamic parameters viz., K_{d}, enthalpy change (ΔH), entropy change (ΔS), Gibbs free energy change (ΔG), and TAS (T being the temperature) were obtained by fitting the isotherms (FIG. 2C and S2, wherein Left panel shows the raw heat data acquired from successive injections of ZnCl₂ into the compound solution. Right panel depicts the integrated binding isotherms as a function of the binary complex at 298 K. Fitting was obtained from the data points with one set of the binding site model), wherein Left panel shows the raw heat data acquired from successive injections of Zn into the compound solution. Right panel depicts the integrated binding isotherms as a function of the binary complex at 298 K. Fitting was obtained from the data points with one set of the binding site model). The ITC studies revealed 1c to be the best chelator of Zn with K_{d} value of 29.06±7.5 μM followed by 1b and 1a with K_{d} value of 50.4±13.7 μM and 2.8±0.8 mM, respectively. The K_{d} values determined from ITC validate the results obtained from other binding studies discussed vide supra (lower K_{d} and higher K_{SV}). Both endothermic and exothermic peaks were observed upon binding of 1c and Zn resulting in a net negative ΔH value, and the data was fitted to exothermic one site binding model. For a reaction to be spontaneous, it must be favored by at least one of the factors, enthalpy or entropy. The binding between 1c and Zn was found to be favored by both enthalpy (ΔH=−0.698±0.223 kcal mol⁻¹) and entropy (−TΔS=−5.75 kcal mol⁻¹) with later being the predominant factor. The resulting ΔG value was found to be −6.45 kcal mol⁻¹, implying that binding of 1c to Zn is a spontaneous process and occurs without the intervention of any external force. A similar trend was observed in case of 1b where the spontaneity of binding (ΔG=−5.78 kcal mol⁻¹) is the result of both enthalpy (−0.551±0.056 kcal mol⁻¹) and entropy (−TΔS=−5.23 kcal mol-1) contributions, wherein the predominant factor entropy was associated with hydrophobic binding interaction. In case of 1a, the enthalpy factor was found to have favorable effect (ΔH=−13.1±0.13), while unfavorable entropic contribution (−TΔS=9.59) resulted in ΔG of −3.48 kcal/mol. Table 1 illustrates that the binding parameters obtained from absorbance, fluorescence and ITC studies demonstrated that 1c is better chelator.

TABLE 1
Binding constants and energy parameters
of small molecules with Zn.

| | Compounds | | |
|---|---|---|---|
| Binding parameters | 1a* | 1b | 1c |
| Benesi-Hildebrand K_{d}(mM) | 38.3 | 22.9 | 20.2 |
| Stern-Volmer binding constant (K_{SV}) | 0.06 × | 1.7 × | 1.7 × |
| | 10⁴ | 10⁴ | 10⁴ |
| ITC K_{d} | 2.8 ± | 50.4 ± | 29.06 ± |
| | 0.8 | 13.7 | 7.5 |
| ΔH | −13.1 ± | −0.551 ± | −0.693 ± |
| | 0.13 | 1 | 1 |
| | | 0.056 | 0.018 |
| −TΔS | 9.59 | −5.23 | −5.75 |
| ΔG | −3.48 | −5.78 | −6.45 |

### Example 5: Interaction of Small Molecules with Tau Protein

Tau undergo LLPS by homotypic interactions and small molecules binding to tau can potentially interfere with tau-tau interaction to modulate LLPS. Small molecules with CDP containing aromatic a-side chain, NMI and DPA were expected to strongly bind to tau. The ability of small molecules to bind with tau was evaluated by tau intrinsic fluorescence assay and the molecular level interaction was understood by docking studies.

### Example 5.1: Intrinsic Fluorescence Assay

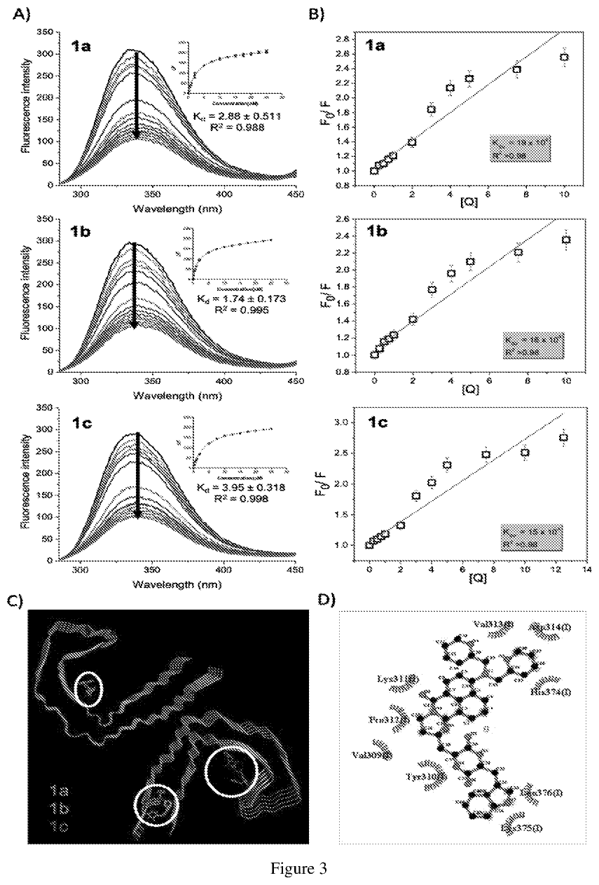

The binding of small molecules with tau protein was monitored using tau intrinsic fluorescence spectral changes. Upon addition of compounds, the intrinsic emission band of tau at ˜330 nm showed quenching whereas compounds exhibited enhanced fluorescence at 530 nm (FIG. 3A and S3). The change in fluorescence intensity was fitted with one site binding saturation model to determine the K_{d} values. The K_{d} values of 1a, 1b and 1c with tau were found to be 2.88±0.51 μM, 1.74±0.17 μM, and 3.95±0.32 μM, respectively (FIG. 3A inset) which implied strong binding of small molecules with tau. The K_{SV} values were obtained from the Stern-Volmer equation to further quantify the extent of binding. The K_{SV} values were found to be 19×10⁴ M⁻¹, 16×10⁴ M⁻¹ and 15×10⁴ M⁻¹ for 1a, 1b and 1c, respectively, which implied that all three small molecules bind strongly to tau with comparable affinity (FIG. 3B). This further emphasized the significance of the skeletal template of the modulators for tau interaction, rather than individual substituted aromatic amino acids.

### Example 5.2: Molecular Docking

Lower K_{d} and higher K_{SV} from the binding studies have demonstrated the strong interaction of small molecules with tau. To gain deeper insights into the molecular interaction of small molecules with tau, molecular docking studies were performed using tau protofibrils (PDB ID: 5O3T of www.rscb.org) and the energy-optimized structures of small molecules. An analysis of the docking results showed that each of the small molecules bind to tau at different and distinct sites as depicted in FIG. 3C. This was attributed to the fact that although all small molecules possess similar structure, their preferred orientation in the energy optimized state as obtained from Gaussian energy minimization and in the protein domains after docking were very different from each other. The Gibbs free energy or ΔG value of tau-molecules binding was negative in all cases, indicating a thermodynamically favored spontaneous binding of the small molecules to tau protofibril. The lowest value of ΔG was seen in case of 1c (−34.1 kJ/mol), followed by 1a (−33.3 kJ/mol) and 1b (−32.9 kJ/mol). Thus, with the minimum energy value, 1c emerged as the lead candidate with high affinity binding to tau. This trend of binding corroborates well with that obtained from fluorescence measurements and confirm the strong binding of small molecules with tau. 1c with the highest interaction with tau clamped itself within a hydrophobic pocket of chain I of F-J module, whereas 1a and 1c were docked in different chains of A-E module, as depicted in FIG. 3C.

The next part of the docking study consisted of identification of the exact interactions of small molecules with amino acid residues of tau protein. A 2D interaction map was generated to understand the interaction of small molecules with the nearest neighboring residues of tau. In all cases, hydrophobic interactions were found to dominate over hydrogen bonding interactions (FIG. 3D and S4). In case of the strongest binder 1c, electrostatic interactions in the form of hydrogen bonding were not observed within the cut-off distance of 5 Å, while it exhibited numerous hydrophobic interactions with different residues of chain I. The interacting residues of tau were found to be Val309, Tyr310, Lys311, Pro312, Val313, Asp314, His374, Lys 375 and Leu376. It was found that 1c engaged in hydrophobic interaction with His374 of tau which was reported to be involved in Zn binding interaction. In case of 1a, the interactions were predominantly with residues from the C and E chains of the protein. In addition to the hydrophobic interactions, 1a also exhibited two hydrogen bonds with Lys 253 (C) and Lys 363 (E). Similarly, compound 1b also showed a hydrogen bond with His 362 (H), while the residues with which it exhibited hydrophobic interactions span across different chains of the protein. Table 2 shows the inferences obtained from in silico modelling along with fluorescence studies reiterate that the small molecules bind strongly to tau at a molecular level implying that judicious design of small molecules could augment multi-pronged approach to target tau LLPS.

TABLE 2
Binding constants and energy parameters
of small molecules with tau protein.

| | Compounds | | |
|---|---|---|---|
| Binding parameters | 1a | 1b | 1c |
| One site binding saturation fit K_{d} (μM) | 2.88 ± | 1.74 ± | 3.95 ± |
| | 0.51 | 0.17 | 0.32 |
| Stern-Volmer binding constant (K_{SV}) | 19 × | 16 × | 15 × |
| | 10⁴ | 10⁴ | 10⁴ |
| ΔG from docking (kJ/mol) | −33.3 | −32.9 | −34.1 |

### Example 6

### Small Molecules Synergistically Modulate Zn Induced Tau LLPS

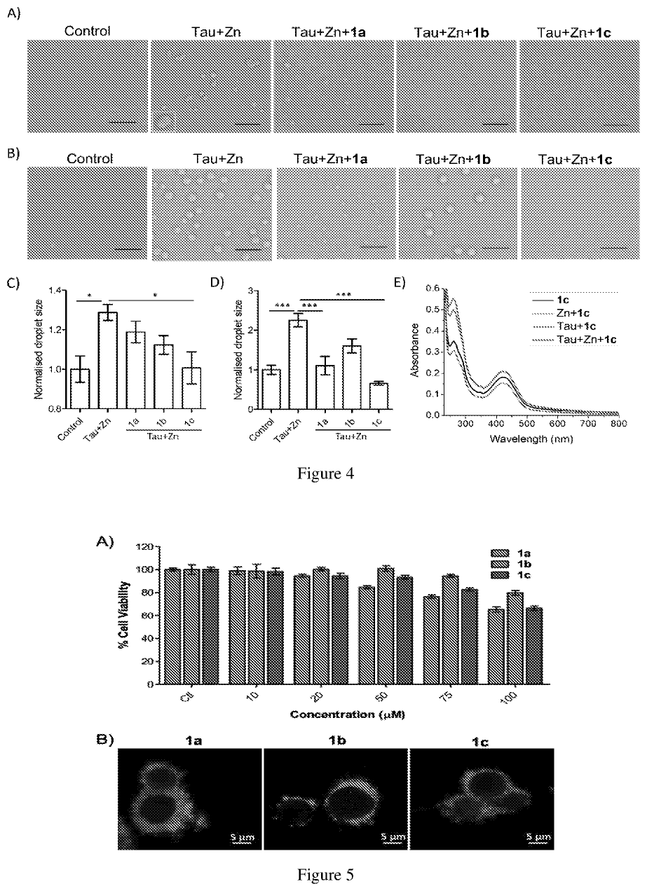

The strong binding interaction of small molecules with Zn and tau encouraged us to evaluate them for modulation of Zn indued LLPS of tau. The ability of small molecules to inhibit tau LLPS and formation of droplets were studied by differential interface contrast (DIC) microscopy (FIG. 4). In the presence of Zn, tau readily undergoes LLPS and formed liquid like droplets within 10 min, as evident from the observed fusion droplets (FIG. 4A and inset). The samples incubated with small molecules inhibits LLPS of tau as evident from DIC imaging, which revealed reduction in droplets and droplet size as tau failed to phase separate to form condensates. Among small molecules 1c showed highest inhibitory effect owing to its superior binding towards both Zn and tau (FIG. 4A). Analysis of droplets size (normalized to control) revealed droplet size of ˜1.3 fold in case of Zn induced LLPS samples, whereas droplets size reduced to ˜1.19 and ˜1.12-fold for 1a and 1b treated samples, respectively. Small molecules 1c exhibited highest inhibitory effect that reduced droplet number and size comparable to control (˜1) (FIG. 4C). The inhibitory effect is in the order of 1c followed by 1b and 1a, which agrees with Zn and tau binding ability of small molecules. The LLPS of tau is through transient weak interactions and we believe that the small molecules with strong multivalent interactions with tau and Zn (chelation) potentially dissolve the phase separated droplets. To evaluate the reversal effect of small molecules on phase separated droplets, tau LLPS was induced (10 min) and treated with small molecules for 15 min followed by visualization by DIC imaging. DIC micrographs showed that small molecules exhibit dissolution effect evident from reduction in number and size of condensates (FIG. 4B). The quantification of droplets size revealed the increased size in Zn treated samples to 2.26-fold, which reduced to ˜1.1 and ˜1.6-fold for 1a and 1b treated samples, respectively (FIG. 4D). The droplet size was reduced to less than control in case of 1c treated samples (˜0.66-fold) indicating its ability to inhibit Zn induced tau LLPS. The modulatory effect of 1c prompted to look for its preferential interaction among tau and Zn. UV-Visible spectroscopy was used to understand preferential binding of 1c with Zn and tau. The absorption spectra of 1c in the presence of tau and Zn (independently) and together was recorded. It was observed that binding of 1c with tau and Zn results in hyperchromic and hypochromic shifts, respectively, which indicates differential interactions (FIG. 4E). Small molecule 1c exhibited hyperchromic shift in the presence of both tau and Zn indicating preferential interaction of 1c with tau over Zn, which agreed with the determined binding constants. The preferential binding of small molecule 1c to tau over Zn infer the possible binding of small molecules to tau followed by inhibition of tau-tau interactions, interfering with the Zn coordination to tau, and chelate Zn that synergistically modulate the tau LLPS.

### Example 7

### Small Molecules are Biocompatible and Cell Permeable

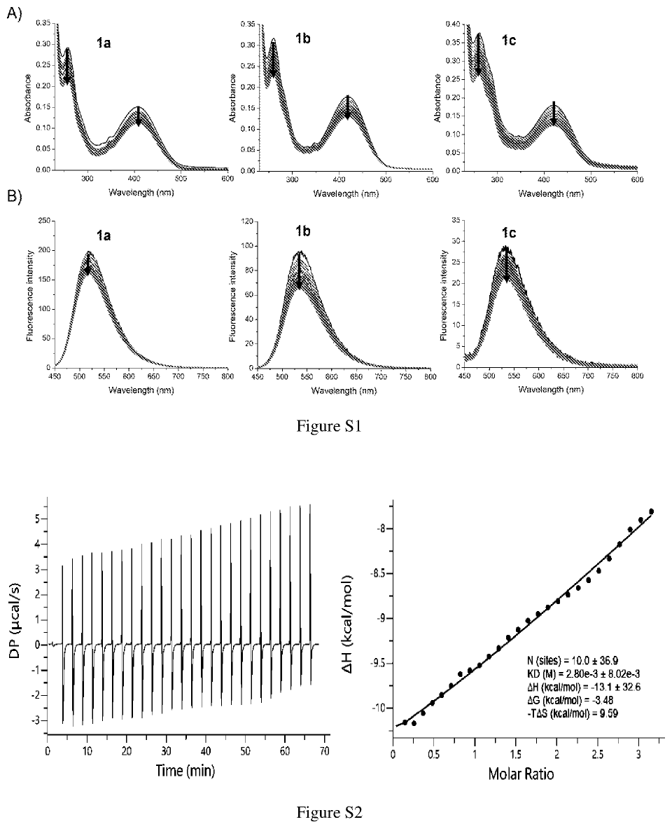

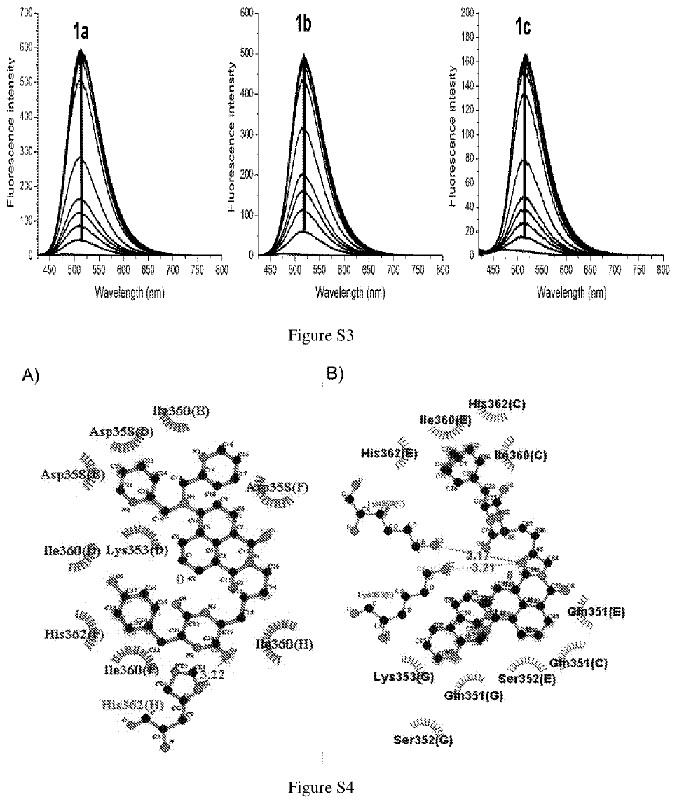

The potential role of small molecules in combating LLPS motivated us to evaluate their biocompatibility. The small molecules with multivalent interactions can be toxic and impermeable to cells, which may limit their utility as therapeutic agents. The cytotoxicity of small molecules was evaluated in SH-SY5Y cells by MTT assay. Cells were treated with different concentration of compounds (10 to 100 μM) and incubated for 24 h to evaluate their cytotoxic effect. The results showed that compounds 1a-c are non-toxic up to 20 μM, while 1b and 1c exhibit excellent compatibility up to 50 μM (FIG. 5A). To inhibit intracellular tau LLPS, compounds need to cross the plasma membrane and accumulate inside the cells. The inherent fluorescence of small molecules (1a-c) were assessed their cell permeability by fluorescence microscopy imaging. SH-SY5Y cells were treated with 10 μM of compounds and incubated for 1 h and subjected to live cell fluorescence imaging. Small molecules exhibited good cellular uptake, as the microscopy images showed their distribution across the cytoplasm underscoring the potential of small molecules to modulate LLPS in cellular milieu (FIG. 5B). The biocompatibility up to 50 μM and effective cell permeability reiterate the potential of small molecules for in-cellulo and in vivo modulation of tau LLPS.

LLPS regulates many cellular physiological processes. Recent findings have clearly demonstrated the role of LLPS in disease conditions like cancer and neurodegenerative disorders. Understanding the chemical interactions (chemical grammar) that drive the LLPS and factors that influence the complex process shed light on designing the chemical modulators of LLPS. The present disclosure has demonstrated the novel approach of integrating diverse chemical moieties to rationally design potent small molecules of LLPS. The compounds of the present disclosure counteract the molecular interaction that drives Zn mediated tau LLPS. Among the designed molecules, compound 1c with strong Zn and tau binding ability emerged as the potent modulator with both inhibition of LLPS and dissolution of phase separated condensates. The approach set the platform to develop novel small molecules targeting various biomolecules that undergo LLPS implicated in different disease conditions by understanding their chemical grammar. Tau undergoes aggregation through phase separated condensate that reiterates targeting tau LLPS is tangible drug development approach, small molecules 1c effectively modulates the tau LLPS and has potential implications in targeting tau toxicity. The compounds of the present disclosure laid the foundation for designing novel small molecules that target tau LLPS to develop future therapeutics for AD and other tauopathies.

### ADVANTAGES OF THE PRESENT DISCLOSURE

The above-mentioned implementation examples as described on this subject matter and its equivalent thereof have many advantages, including those which are described.

The present disclosure provides potent cyclic dipeptide small molecules by integrating multiple chemical groups that offer diverse chemical interactions to modulate tau LLPS. The compounds of the present disclosure effectively inhibit and dissolve Zn mediated tau LLPS condensates. This approach of developing small molecule modulators of LLPS establish a novel platform that has potential implication for the development of therapeutics for cancer and neurodegenerative disorders.

Although the subject matter has been described in considerable detail with reference to certain embodiments thereof, other embodiments are possible. As such, the spirit and scope of the disclosure should not be limited to the description of the embodiments contained herein.

I/We claim:

1. A compound of Formula (I) 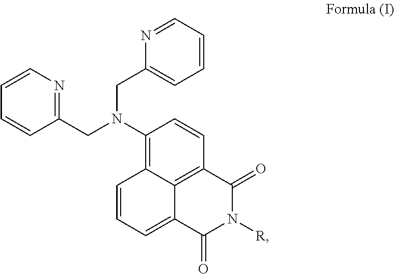
its stereoisomers, intermediates, or pharmaceutically acceptable salts thereof,
wherein
R is C₁₋₁₀ alkyl substituted with a group selected from C₂₋₁₀ heterocyclyl, C₆₋₁₀ aryl, or C₂₋₁₀ heteroaryl, wherein C₂₋₁₀ heterocyclyl, C₆₋₁₀ aryl, or C₂₋₁₀ heteroaryl is optionally substituted with one or more substituents selected from hydrogen, hydroxyl, carboxyl, oxo, CONH₂, NH₂, C₁₋₁₀ alkyl, C₁₋₆ alkylamine, halogen, C₁₋₆ haloalkyl, cyano, or R¹,
R¹ is selected from C₁₋₁₀ alkyl, C₁₋₆ alkylamine, or C₁₋₆ haloalkyl, wherein C₁₋₁₀ alkyl, C₁₋₆ alkylamine, or C₁₋₆ haloalkyl is optionally substituted with C₆₋₁₀ aryl or C₂₋₁₀ heteroaryl, and wherein C₆₋₁₀ aryl or C₂₋₁₀ heteroaryl is optionally substituted with one or more substituents selected from hydroxyl, carboxyl, oxo, CONH₂, NH₂, halogen, or cyano.

2. The compound of Formula (I), its stereoisomers, intermediates, or pharmaceutically acceptable salts thereof as claimed in claim 1, wherein the compound is selected from
(i) 2-(4-((2S,5S)-5-benzyl-3,6-dioxopiperazin-2-yl)butyl)-6-(bis(pyridin-2-ylmethyl)amino)-1H-benzo[de]isoquinoline-1,3(2H)-dione of Formula (Ia);
(ii) 6-(bis(pyridin-2-ylmethyl)amino)-2-(4-((2S,5S)-5-(4-hydroxybenzyl)-3,6-dioxopiperazin-2-yl)butyl)-1H-benzo[de]isoquinoline-1,3(2H)-dione of Formula (Ib); or
(iii) 2-(4-((2S,5S)-5-((1H-indol-3-yl)methyl)-3,6-dioxopiperazin-2-yl)butyl)-6-(bis(pyridin-2-ylmethyl)amino)-1H-benzo[de]isoquinoline-1,3(2H)-dione of Formula (Ic).

3. The compound of Formula (I), its stereoisomers, intermediates, or pharmaceutically acceptable salts thereof, as claimed in any one of the claims 1 to 2, wherein the compound is for use in the manufacture of a medicament for treating a neurodegenerative disease selected from Alzheimer's disease (AD), Pick's disease, tauopathies, frontotemporal dementia associated with tau-immunoreactive inclusions (FTD-tau), progressive supranuclear palsy (PSP), corticobasal degeneration (CBD), chronic traumatic encephalopathy, primary age related tauopathy, argyrophilic grain disease, or post-encephalitic parkinsonism.

4. The compound of Formula (I), its stereoisomers, intermediates, or pharmaceutically acceptable salts thereof as claimed in any one of the claims 1 to 3, wherein the compound modulates aggregation of tau.

5. The compound of Formula (I), its stereoisomers, intermediates, or pharmaceutically acceptable salts thereof as claimed in any one of the claims 1 to 4, wherein the compound is capable of binding with Zn²⁺ ion and tau protein.

6. The compound of Formula (I), its s stereoisomers, intermediates, or pharmaceutically acceptable salts thereof as claimed in any one of the claims 1 to 5, wherein the compound modulates tau liquid-liquid phase separation.

7. The compound of Formula (I), its stereoisomers, intermediates, or pharmaceutically acceptable salts thereof as claimed in any one of the claims 1 to 6, wherein the compound is capable of inhibition or dissolution of Zinc mediated liquid-liquid phase separated condensate and aggregates of tau.

8. A chelate complex comprising a chelating ligand comprising the compound of Formula (I), its stereoisomers, intermediates, or pharmaceutically acceptable salts thereof as claimed in any one of the claims 1 to 7, with one or more metal ions; and, optionally, a binding moiety of tau protein.

9. The chelate complex as claimed in claim 8, wherein the chelate complex comprises the compound of Formula (I), its stereoisomers, intermediates, or pharmaceutically acceptable salts thereof as claimed in any one of the claims 1 to 7, with Zn²⁺ as metal ions; and, optionally, a binding moiety of tau protein.

10. A method for the preparation of the compound of Formula (I), its stereoisomers, intermediates, or pharmaceutically acceptable salts thereof as claimed in any one of the claims 1 to 7, the method comprising:
a. reacting a compound of Formula II and a compound of Formula III in the presence of a base and a solvent; and incubating to form a compound of Formula (I), 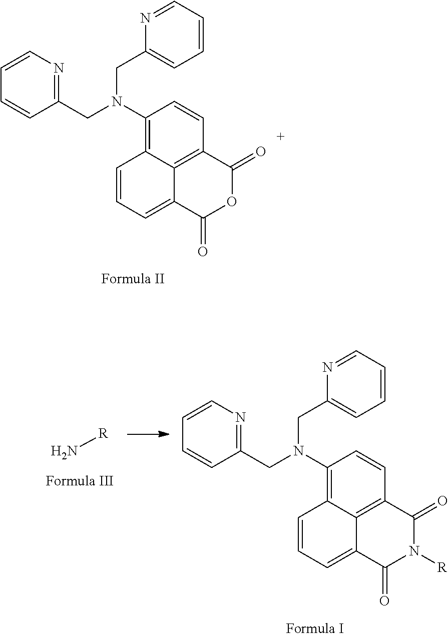
wherein
R is C₁₋₁₀ alkyl substituted with a group selected from C₂₋₁₀ heterocyclyl, C₆₋₁₀ aryl, or C₂₋₁₀ heteroaryl, wherein C₂₋₁₀ heterocyclyl, C₆₋₁₀ aryl, or C₂₋₁₀ heteroaryl is optionally substituted with one or more substituents selected from hydrogen, hydroxyl, carboxyl, oxo, CONH₂, NH₂, C₁₋₁₀ alkyl, C₁₋₆ alkylamine, halogen, C₁₋₆ haloalkyl, cyano, or R¹,
R¹ is selected from C₁₋₁₀ alkyl, C₁₋₆ alkylamine, or C₁₋₆ haloalkyl, wherein C₁₋₁₀ alkyl, C₁₋₆ alkylamine, or C₁₋₆ haloalkyl is optionally substituted with C₆₋₁₀ aryl or C₂₋₁₀ heteroaryl, and wherein C₆₋₁₀ aryl or C₂₋₁₀ heteroaryl is optionally substituted with one or more substituents selected from hydroxyl, carboxyl, oxo, CONH₂, NH₂, halogen, or cyano.

11. The method for the preparation of the compound of Formula (I), its stereoisomers, intermediates, or pharmaceutically acceptable salts thereof as claimed in claim 10, wherein said incubation is performed at a temperature in a range of 100 to 120 degree Celsius for a period in a range of 4 to 16 hours.

12. The method for the preparation of the compound of Formula (I), its stereoisomers, intermediates, or pharmaceutically acceptable salts thereof as claimed in claim 10, wherein the base is selected from triethylamine, diisopropylethylamine, 1,8-diazabicyclo(5.4.0) undec-7-ene, pyridine, or combinations thereof; and the solvent is selected from dimethylformamide, dimethylsulphoxide, isopropyl alcohol, or combinations thereof.

13. The method for the preparation of the compound of Formula (I), its stereoisomers, intermediates, or pharmaceutically acceptable salts thereof as claimed in claim 10, wherein the method further comprises purifying the compound of Formula (I), by centrifuging to obtain a precipitate and washing the precipitate to obtain of the compound of Formula I.

14. A pharmaceutical composition comprising the compound of Formula (I) its stereoisomers, intermediates, or pharmaceutically acceptable salts thereof as claimed in any of claims 1 to 7, with a pharmaceutically acceptable carrier, optionally in combination with one or more other pharmaceutical compositions.

15. The pharmaceutical composition as claimed in claim 14, wherein the composition comprises the compound of Formula (I), its stereoisomers, intermediates, or pharmaceutically acceptable salts thereof as claimed in any of claims 1 to 7, wherein the composition is in a form selected from tablet, capsule, powder, syrup, solution, aerosol, or suspension.
